(19) United States
(12) Patent Application Publication
(10) Pub. No.: US 2012/0209162 A1
(43) Pub. Date: Aug. 16, 2012

(54) PELVIC SPLINT

(75) Inventors: Bernard Bar-Natan, Givataim (IL); Michal Stadler Bekerman, Gedera (IL); Amnon Hamdani, Ashdod (IL); Graham Hill, London Greater London (GB)

(73) Assignee: FIRST CARE PRODUCTS LTD., Lod (IL)

(21) Appl. No.: 13/502,726

(22) PCT Filed: Oct. 28, 2010
(86) PCT No.: PCT/IL10/00896

§ 371 Date: Apr. 18, 2012

(30) Foreign Application Priority Data
Oct. 29, 2009 (IL) 201848

(51) Int. Cl. A61F 5/00

(52) U.S. Cl. 602/19

(57) ABSTRACT

There is provided herein a fastenable pelvic splint, comprising: (a) a buckle comprising a contractible ratchet mechanism and a fastening handle; and (b) a flexible band adapted to be connected to the buckle at two opposite ends of the buckle and to circumscribe the pelvis. The fastenable pelvic splint may be adapted to be fastened around the pelvic by contracting the distance between the two opposite ends of the buckle using the ratchet mechanism.

### FIELD OF THE INVENTION

The invention relates to pelvic splints. Some embodiments of the invention relate to pelvic splint with rapid tightening and loosening mechanism.

### BACKGROUND OF THE INVENTION

External pelvic splints are effective for immobilizing pelvic or hip fractures and also for reducing haematoma and bleeding in cases of abdominal injuries. Pelvic splints are commonly used in emergency medicine, for example in accident scenes, battle field, etc.

Treatment for hip fractures is described, for example, in M Tile (Ed.), D. L. Helfet (Edi.) and J. F. Kellam (Ed.), Fractures of the Pelvis and Acetabulum, Lippincott Williams & Wilkins, ISBN-13: 9780781732130. Devices for hip or pelvis stabilization are described, for example, in US Application 2007/0197945 that discloses a hip and pelvic splint system designed to immobilize both the pelvic region and lower extremities of a subject suffering from a hip or pelvic fracture. U.S. Pat. No. 503217 and U.S. Pat. No. 6,793,639 patents disclose pelvic splints for immobilization and maintaining the annular integrity of the pelvis with two or more adjustable straps.

### SUMMARY OF THE INVENTION

An aspect of the invention relates to a pelvic splint rapidly tightenable by one hand operation (of a care giver or a subject in a need of the pelvic splint). According to some embodiments, the pelvic splint may be loosened by a release trigger.

The pelvic splint (hereinafter ‘splint’) is designed for rapid fitting on a subject having pelvic trauma such as fracture or shock damage because a quick immobilization may be crucial for resuscitation and impeding deterioration such as haematoma. Likewise, the splint is designed for an instant release as the subject may need emergency treatment wherein a splint could be obstructive. Additionally, the splint is designed to be transparent for x-ray so that the subject can be examined while stabilized.

In some embodiments, the splint comprises two distinctive components: (a) a buckle having a releasable latching ratchet mechanism and (b) a flexible band having linkage formations about the length thereof and connected by linkage formations to the buckle about two opposite ends thereof forming a circumference of the splint.

The buckle comprises (a) a handle that upon turning contracts the buckle's widths between two opposite ends thereof, pulling the band and reduces the splint's circumference thereby tightening the band about the subject and retaining the tight position by the latched ratchet (locked and closed position), and (b) a trigger that upon activating thereof releases the ratchet thereby loosening the band about the subject (open position).

In some embodiments, the ratchet mechanism comprises two teethed elongated members (‘rods’) parallely mounted and movable relative to each other. By rotating the handle the rods move unidirectionally closer to each other increasing the overlap extent therebetween, optionally against an elastic force, thus contracting the distance between the buckle ends. The rods are restrained from moving apart by the latching (locking) of the ratchet mechanism. Setting off the trigger releases the latch of the ratchet mechanism and the rods are freed, optionally recoiling back under the optional elastic force.

In some embodiments, the trigger is formed as a knob or handle or any other appendage or grip which is activated such as by pushing, pulling, sliding or rotating. In some embodiments, the trigger is integrated in the handle, such as by a pushdown knob, for example about the handle axis. Optionally or alternatively, the handle constitutes also the trigger which is activated such as by pulling (or pushing) the handle or, alternatively, rotating the handle in a counter direction to the tightening direction.

In some embodiments, the splint is provided with separated buckle and band and ready for assembly on the subject. As one assembly possibility the band is wrapped about the pelvis of the subject and one or more linkage formations are connected to one end of the buckle and one or more linkage formations farther along the band's length are connected to the opposite end of the buckle. As another assembly possibility one or more linkage formations of the band are connected to one end of the buckle and after the band is wrapped about the subject one or more linkage formations farther along the band's length are connected to the opposite end of the buckle.

In some embodiments, the splint is provided with the buckle already connected at one end thereof to the band by linkage formations thereof. For assembly on the subject the band is wrapped about the pelvis of the subject and one or more linkage formations farther along the band's length are connected to the opposite end of the buckle.

In some embodiments, after fixing the splint on the subject the possible remainder loose end of the band is fastened to the wrapped band by one or more linkage formations along the band's length.

In some preferred embodiments of the invention, the band is foldable and optionally provided tightly folded together with the buckle.

Another aspect of the invention relates to a method for stabilizing a subject by a flexible band by releasable tightening the band by a handle that contracts a buckle having a releasably locking ratchet mechanism. The stabilization is preferably carried out by one motion optionally with one hand.

In some embodiments, the splint is suitable or adapted to other parts or limbs of a subject, optionally by adapting the length of the band and/or size of the buckle.

In some embodiments, the splint is provided as a kit comprising at least one buckle and at least one band or as a plurality of buckles and bands. Optionally the bands are of various lengths and/or the buckles are of various sizes for different kinds of subjects and/or organs and/or limbs.

In the specification and claims the following terms and derivatives and inflections thereof imply the respective non-limiting characterizations below, unless otherwise specified or evident from the context.

A subject may be according to some embodiments, a person, a subject, a wounded person or an injured person.

A ratchet may be according to some embodiments, a mechanism comprising a bar or a wheel (or other structure) having teeth into which a pawl (latch) drops or pushes so that motion can be imparted to the wheel or bar to allow effective motion in one direction only.

A buckle may be according to some embodiments, a device for holding together ends of a band or belt.

Linkage formation(s) may be according to some embodiments, structure(s) and/or opening(s) adapted for connection with another article typically by corresponding (mating, coupling) structures in the latter (for brevity also referred to as ‘formations’).

Mushroom-like button(s) may be according to some embodiments, structure(s) formed as a stem and a structure larger in width or diameter than the stem about an end thereof (also referred to as mushroom button).

A handle may be according to some embodiments, a structure for operating a mechanism by a hand, such as a lever or slider.

According to an aspect of some embodiments of the present invention there is provided a fastenable pelvic splint, comprising:

a buckle comprising a contractible ratchet mechanism and a fastening handle; and

a flexible band adapted to be connected to the buckle at two opposite ends of the buckle and to circumscribe the pelvis.

In some embodiments, the splint is adapted to be fastened around the pelvic by contracting the distance between the two opposite ends of the buckle using the ratchet mechanism.

In some embodiments, the splint is adapted to be fastened by a motion of the handle and wherein the handle is operable using a single hand.

The splint according to claim 1, wherein the buckle further comprises a locking mechanism adapted to lock the splint in a fasten position.

In some embodiments, the buckle further comprises a release trigger adapted to loosen the splint.

In some embodiments, the band is adapted to be removably connected to the buckle.

In some embodiments, the band comprises linkage formations along at least a portion of the length of the band, wherein the linkage formations are adapted to connect the band to the buckle.

In some embodiments, the buckle comprises structures adapted to mate with the linkage formations of the band.

In some embodiments, the buckle comprises buttons and wherein the linkage formations of the band comprise slits, wherein the buttons are adapted to fit to the slits thereby connect the band to the buckle.

In some embodiments, the splint is substantially x-ray transparent.

The splint according to claim 1, wherein the splint is disposable.

According to an aspect of some embodiments of the present invention there is provided a kit for a pelvic splint, comprising:

at least one buckle comprising a contractible ratchet mechanism and a fastening handle; and

at least one flexible band adapted to be connected to the buckle at two opposite ends of the buckle and to circumscribe the pelvis.

In some embodiments, the splint is adapted to be fastened around the pelvic by contracting the distance between the two opposite ends of the buckle using the ratchet mechanism.

In some embodiments, the splint is adapted to be fastened by a motion of the handle and wherein the handle is operable using a single hand.

In some embodiments, the buckle further comprises a locking mechanism adapted to lock the splint in a fasten position.

In some embodiments, the buckle further comprises a release trigger adapted to loosen the splint.

In some embodiments, the band is adapted to be removably connected to the buckle.

In some embodiments, the band comprises linkage formations along at least a portion of the length of the band, wherein the linkage formations are adapted to connect the band to the buckle.

In some embodiments, the buckle comprises structures adapted to mate with the linkage formations of the band.

In some embodiments, the buckle comprises buttons and wherein the linkage formations of the band comprise slits, wherein the buttons are adapted to fit to the slits thereby connect the band to the buckle.

In some embodiments, the splint is substantially x-ray transparent.

In some embodiments, the splint is disposable.

### BRIEF DESCRIPTION OF THE DRAWINGS

Some non-limiting exemplary embodiments of the invention are illustrated in the following drawings.

Identical or duplicate or equivalent or similar structures, elements, or parts that appear in one or more drawings are generally labeled with the same reference numeral, optionally with an additional letter or letters to distinguish between similar objects or variants of objects, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

It should be noted that some figures were converted to black-and-white rendering, thereby degrading the pictorial quality such as by reducing certain details or texture or fineness.

FIG. 1 schematically illustrates a pelvis splint on a pelvis, according to exemplary embodiments of the invention;

FIG. 2A schematically illustrates a buckle for a pelvis splint in a closed position, according to exemplary embodiments of the invention;

FIG. 2B schematically illustrates a buckle for a pelvis splint in an open position, according to exemplary embodiments of the invention;

FIG. 2C schematically illustrates a mushroom button, according to exemplary embodiments of the invention;

FIG. 2D illustrates operation by hand for tightening and locking a buckle of a splint, according to exemplary embodiments of the invention;

FIG. 3A schematically illustrates a band for a pelvis splint, according to exemplary embodiments of the invention;

FIG. 3B schematically illustrates a linkage formation as opening in a band for a pelvis splint, according to exemplary embodiments of the invention;

FIG. 3C schematically illustrates insertion a mushroom button in a linkage formation as opening in a band for a pelvis splint, according to exemplary embodiments of the invention;

FIG. 3D schematically illustrates fixing a mushroom button in a linkage formation as opening in a band for a pelvis splint, according to exemplary embodiments of the invention;

FIG. 4 schematically illustrates a folded pelvis splint, according to exemplary embodiments of the invention;

FIG. 5A schematically illustrates a partially connected band of a splint is wrapped about a pelvis, according to exemplary embodiments of the invention;

FIG. 5B schematically illustrates folding loose part of a band of a splint after wrapping on a pelvis, according to exemplary embodiments of the invention;

FIG. 5C schematically illustrates fixing loose part of a band of a splint after wrapping on a pelvis, according to exemplary embodiments of the invention;

FIG. 5D schematically illustrates a double-ended mushroom button, according to exemplary embodiments of the invention;

FIG. 6 schematically illustrates a section of a symmetrical band of a splint, according to exemplary embodiments of the invention;

FIG. 7A schematically outlines a sequence of operations for donning a splint on a pelvis, according to exemplary embodiments of the invention;

FIG. 7B schematically outlines another sequence of operations for donning a splint on a pelvis, according to exemplary embodiments of the invention; and

FIG. 8A schematically illustrates a kit for assembling a plurality of pelvis splints of different sizes, according to exemplary embodiments of the invention; and

FIG. 8B schematically illustrates a kit for assembling a plurality of pelvis splints of different sizes using a buckle of same size, according to exemplary embodiments of the invention.

### DESCRIPTION OF EMBODIMENTS OF THE INVENTION

The following description relates to one or more non-limiting examples of embodiments of the invention. The invention is not limited by the described embodiments or drawings, and may be practiced in various manners or configurations or variations. The terminology used herein should not be understood as limiting unless otherwise specified.

The non-limiting section headings used herein are intended for convenience only and should not be construed as limiting the scope of the invention.

### Pelvis Splint

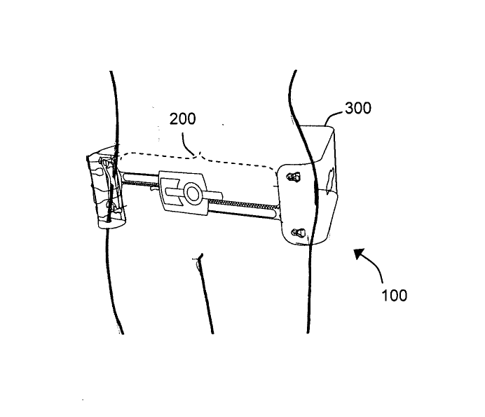

FIG. 1 schematically illustrates a pelvis splint 100 on a pelvis, according to exemplary embodiments of the invention. The splint comprises a buckle 200 (indicated by a dashed bracket and shown in an open position) having a ratchet mechanism on the pelvis and a band 300 wrapped about the pelvis and therefore only partially shown.

Splint 100 is generally used as non-limiting example in the descriptions below.

### Buckle

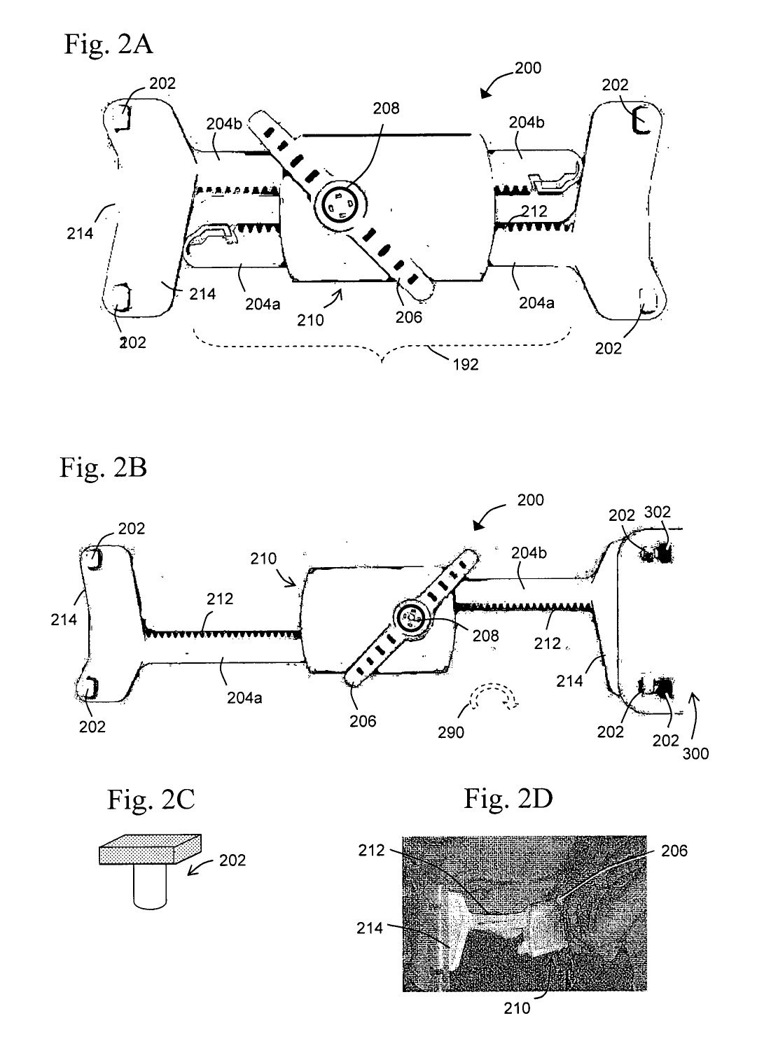

FIGS. 2A-B schematically illustrates a buckle 200 for pelvis splint 100 in closed and open positions, respectively, according to exemplary embodiments of the invention. Buckle 200 has two generally flat opposite side regions (ends) 214. Side regions 214 are further formed or connected with elongated members, or rods, 204 (shown as 204a and 204b) parallel to each other and structured with teeth 212 facing each other. Teethed rods 204 together with (not shown) components inside an enclosure 210, such as a wheel, a latch and an elastic force element, comprise a ratchet mechanism. Buckle 200 further comprises a rotatable handle 206 for contracting and locking (optionally using a locking mechanism, not shown) the ratchet mechanism and a release trigger as pushdown knob 208 for unlocking a locked ratchet mechanism.

By rotating handle 206 in clockwise direction 290 rods 204 move parallely towards each other against an elastic force, increasing the overlap extent between rods 204 (approximately shown by bracket 292) and contracting the distance between opposite side regions 2 14, thereby buckle 200 move s to wards a closed position. Letting go of handle 206 the latch in the ratchet mechanism holds (locks) the mechanism in the current position. By pushing knob 208 the latch is released and rods 204 retreat under the elastic force, decreasing the overlap extent 292 between rods 204 and buckle 200 expands and open.

Buckle 200 further comprises connecting buttons 202 at each side region 214 for connecting a band as described below. Buttons 202 are formed as mushroom buttons as schematically illustrated in FIG. 2C.

Buckle 200 of FIGS. 2A-B is further referenced below as a non-limiting example.

### Band

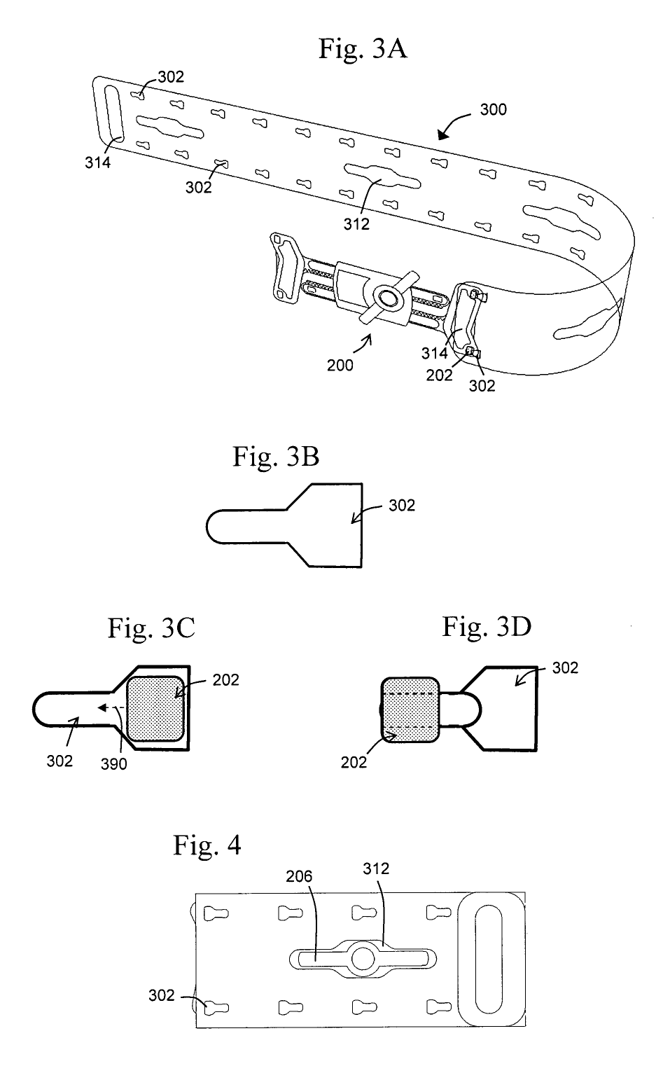

FIG. 2A schematically illustrates a band 300 for pelvis splint 100, according to exemplary embodiments of the invention. Band 300 is formed as a flexible sheet having linkage formations 302 as openings (cuts, slits) shaped with joined wide and narrow sections as schematically illustrated in FIG. 3B.

Linkage formations 302 are used to connect to a side region 214 of buckle 200 by mushroom buttons 202 that mate or couple with formations 302. Inserting button 202 in a wide section of formation 302 and pushing into the narrow section connects and secures band 300 to buckle 200 (akin to a button and loop operation in a garment).

Insertion of mushroom button 202 in a wide section of formation 302 is schematically illustrated in FIG. 3C and pushing into the narrow section of formation 302 is indicated by arrow 390, while FIG. 3D schematically illustrates mushroom button 202 secured in the narrow section of formation 302. It should be noted that connection of band 300 to buckle 200 by buttons 202 and formations 302 is easily removable by sliding band 300 and/or buckle 200 such that buttons 202 are pushed (counter to direction of arrow 390) into the wide section of formations 302 and released.

It is noted that when splint 100 is tightened around a hip of a subject, the tension will help in or cause securing of mushroom buttons 202 in the narrow section of linkage formations 302.

FIG. 2B and FIG. 3A schematically illustrate buckle 200 and band 300 connected as described above, forming a yet open-ended pelvis splint 100.

In some embodiments, band 300 has openings 312 at the length thereof shaped such as to allow handle 206 to push through for folding and un-folding splint 100 as described below. In some embodiments, band 300 has openings 314 about the ends thereof shaped to enable holding or clutching band 300 by a hand for assembly and donning on a subject as described below.

Band 300 of FIG. 3A is further referenced as a non-limiting example.

### Assembly and Donning

In some embodiments, splint 100 is provided in a folded arrangement where band 300 is folded such that openings 314 overlap each other with handle 206 sticking out of openings 314 as schematically illustrated in FIG. 4. In some embodiments, folded splint 100 is further wrapped such as with a tearable paper ring. Optionally, in the folded arrangement buckle band 300 is connected at least to one side region 214 by buttons 202 and linkage formations 302.

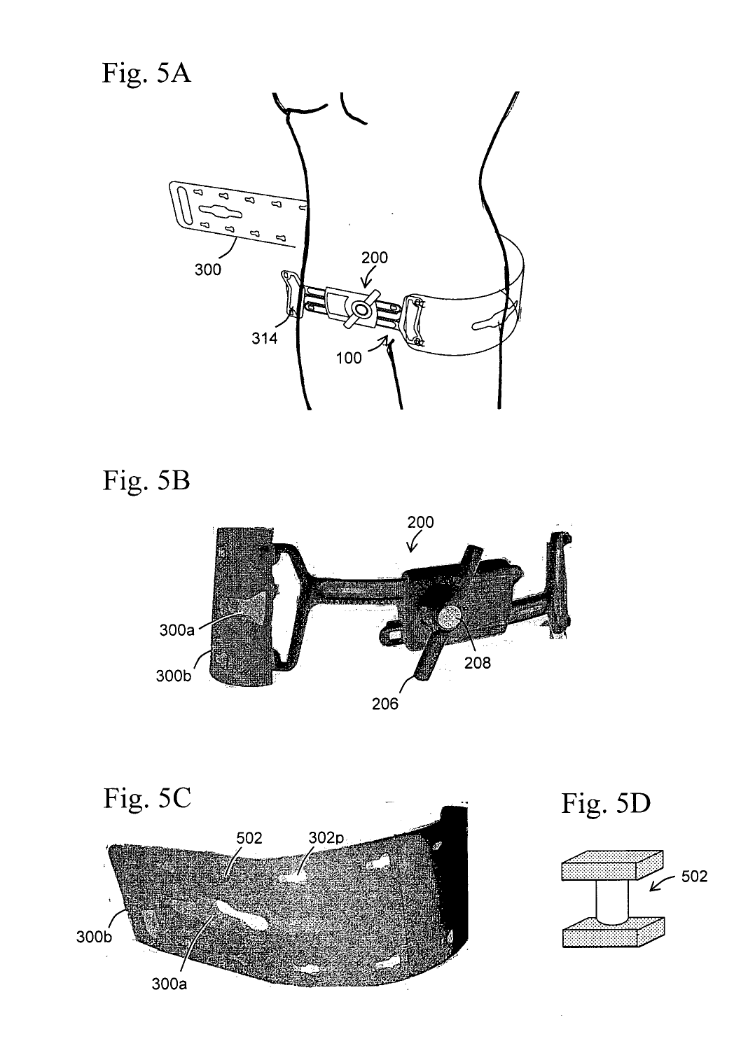

A typical procedure for donning splint 100 on a pelvis is described below. Assuming that band 300 is connected to one side region 214 of buckle 200, as shown, for example, in FIG. 3A. Maintaining buckle 200 in open position band 300 is wrapped around the pelvis of a subject, as illustrated in FIG. 5A, and connected by nearest or nearby linkage formations 302 to buttons 202 on the opposite side region 214 of buckle 200. As illustrated in FIG. 5B, possible remaining loose length of band 300 (slack part) 300b is folded backwards over the wrapped part 300a on the pelvis.

In some cases it is preferred to keep loose part 300b unobstructive to handling splint 100 or the subject. FIG. 5C illustrates how slack part 300b is positioned such that linkage formations 302 overlap at least partially between parts 300a and 300b, and the two parts 300a and 300b are connected via the overlapping formations 302 (e.g. 302p) by a button such as or similar to mushroom button 202, or a double-ended mushroom button 502 as schematically illustrated in FIG. 5D.

Once splint 100 is wrapped on the pelvis handle 206 is rotated clockwise and tightens and locks splint 100 about the pelvis as described above. FIG. 2D illustrates rotation of handle 206 by a hand for contracting and locking buckle 200 of splint 100, according to exemplary embodiments of the invention.

In order to loosen the tightening of splint 100 on the pelvis, trigger knob 208 is pressed releasing the locking of the ratchet mechanism and buckle 200 expands as described above.

### Methods

FIG. 7A schematically outlines a sequence of operations for donning a splint on a pelvis, according to exemplary embodiments of the invention.

An end at one side of band is connected to one end of buckle (702a). The band is wrapped around the pelvis (704b). The other side of the band is connected to the opposite end of the buckle while (moderately) tightening band about the pelvis (706). The splint is tightened and locked by operating (rotating) a handle (708). Subsequently when required the splint is released by activating a trigger (e.g. pushing a knob) (710).

FIG. 7B schematically outlines a variation of the sequence of operations of FIG. 7A, according to exemplary embodiments of the invention.

The band is wrapped around the pelvis (704b) and an end at one side of band is connected to one end of buckle (702a). Operations 706-710 of FIG. 7A ensue in FIG. 7B after operation 702a.

### Some Variations

In some embodiments, splint 100 is tightened by handle 206 having a different shape and buckle 200 optionally closes by rotating in a different direction. Optionally buckle 200 closes and/or locks by another motion such as sliding or pushing or pulling.

In some embodiments, trigger 208 is formed in another shape and optionally operates by different mechanisms such as pulling or pushing a handle (possibly handle 206) or rotating or sliding a knob or lever.

In some embodiments, the linkage formations 302 and corresponding buttons 202 have different shapes. In some embodiments, other methods or fasteners are used to connect band 300 to buckle 200 (and fixing loose end of band 300). For example, loops-and-hooks, pin and socket fasteners or rotating generally flat buttons for securing in narrow section of formation such as 302, or any other fastener.

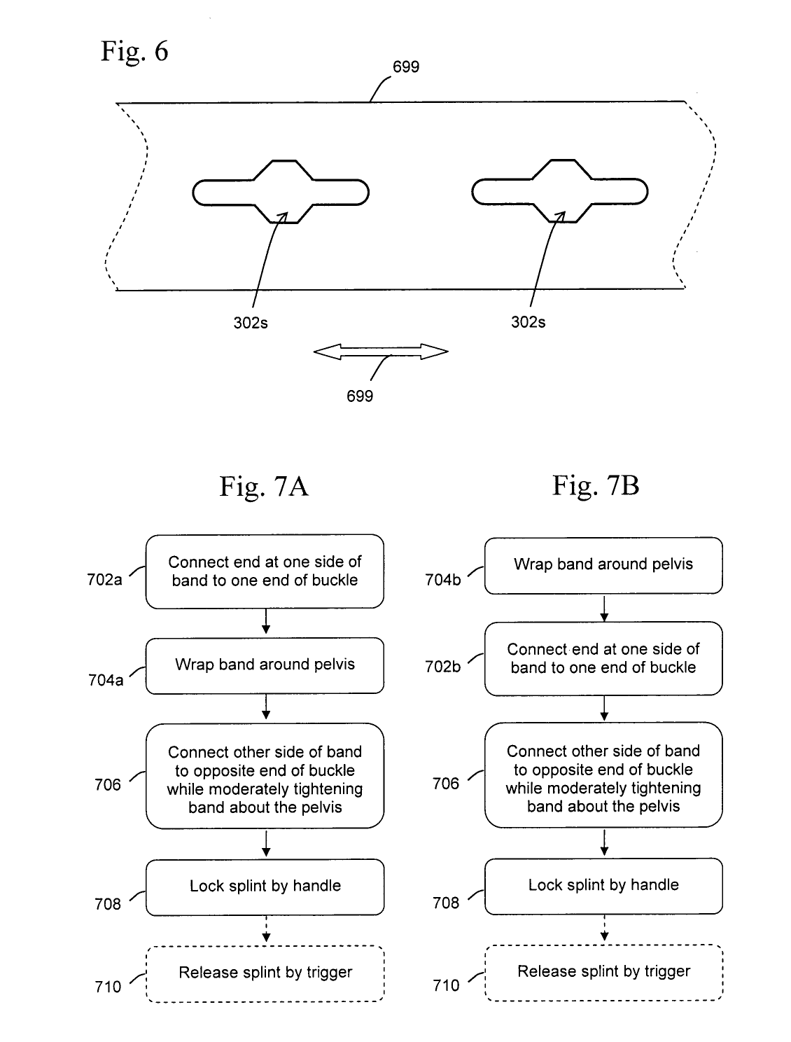

In some embodiments, linkage formations 302 are symmetric (or approximately symmetric) with respect to the length of band 300, such as 302s schematically illustrated in FIG. 6, enabling to connect band 300 to buckle 200 in alternate directions as indicated by double-arrow 699.

In some embodiments, splint 100 or variations thereof is used to stabilize other organs rather than, or in addition to, stabilization of a pelvis, optionally using different sizes of buckle 200 and/or band 300.

In some preferred embodiments of the invention, the band and/or buckle are at least substantially x-ray transparent, allowing to examine the subject by x-ray or CT while the subject is stabilized by the splint.

In some preferred embodiments, the splint, or at least parts thereof, are made of sufficiently inexpensive material and/or by sufficiently inexpensive process (e.g. plastic molding or cutting off-the-shelf plastic sheet) to render the splint affordable to a large spectrum of medical or paramedical services and/or large population, optionally as a disposable article.

### Kit

In some embodiments of the invention, pelvis splint such as splint 100 is provided as a kit, optionally comprising a plurality of bands 300 and/or buckles 200. Optionally bands 300 and/or buckles 200 are of the same or different sizes to fit different pelvises or different organs.

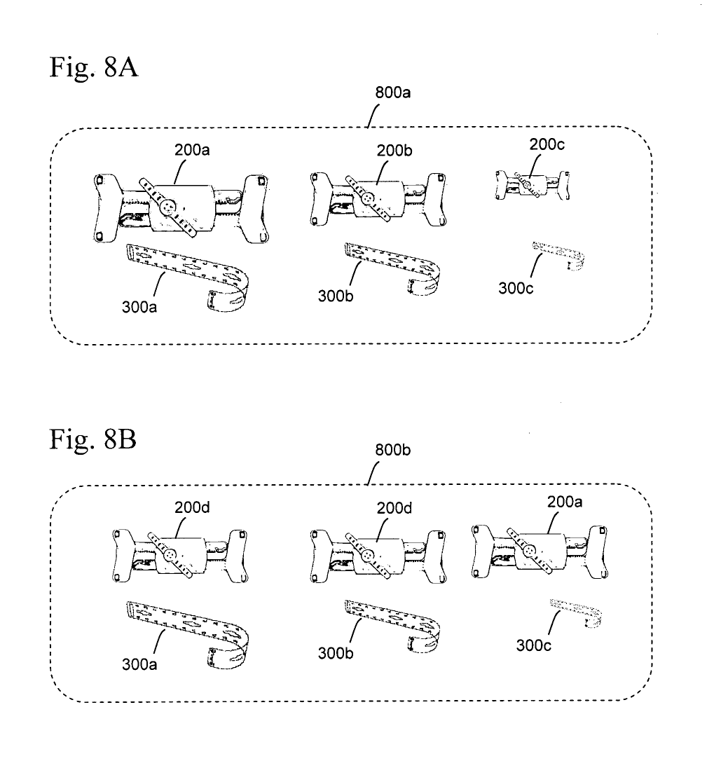

FIG. 8A schematically illustrates a kit 800a (indicated by dotted box) for assembling plurality pelvis splints of different sizes by buckles 200a-c and bands 300a-c, correspondingly, according to exemplary embodiments of the invention.

FIG. 8B schematically illustrates a kit 800b for assembling a plurality of pelvis splints of different sizes using a buckle of same size 200d and bands of different sizes 300a-c, according to exemplary embodiments of the invention.

### Advantages

Potential and probable advantages of the pelvis sprint such as described above are at least (a) rapid fitting on a subject as prompt stabilization for first aid, (b) instant release for accessing the pelvis such as for treatment, and (c) diagnosis by x-ray/CT without having to remove the splint.

### General

The following non-limiting characterizations of terms are applicable in the specification and claim unless otherwise specified or indicated in or evidently implied by the context, and wherein a term denotes also variations, derivatives, inflections and conjugates thereof.

The terms ‘about’, ‘close’, ‘approximate’, ‘practically’ and ‘comparable’ denote a respective relation or measure or amount or quantity or degree yielding an effect that has no adverse consequence or effect relative to the referenced term or embodiment or operation or the scope of the invention.

The terms ‘substantial’, ‘considerable’, ‘significant’ (or synonyms thereof) denote with respect to the context a measure or extent or amount or degree which encompass a large part or most of a referenced entity, or an extent at least moderately or much greater or larger or more effective or more important relative to a referenced entity or with respect the referenced subject matter.

The terms ‘similar’, ‘resemble’, ‘like’ and the suffix ‘-like’ denote shapes and/or structures and/or operations that look or proceed as, or approximately as the referenced object.

The terms ‘constant’, ‘uniform’, ‘continuous’, ‘simultaneous’, ‘equal’ and other seemingly definite terms denote also close or approximate respective terms.

The terms ‘vertical’, ‘perpendicular’, ‘parallel’, ‘opposite’, ‘flat’, ‘straight’ and other angular and geometrical relationships denote also approximate yet functional and/or practical, respective relationships.

The terms ‘preferred’, ‘preferably’, ‘typical’ or ‘typically’ do not limit the scope of the invention or embodiments thereof.

The terms ‘exemplary’ or ‘example’ denote a non-limiting illustration and do not limit the scope of the invention or embodiments thereof.

The terms ‘comprises’, ‘comprising’, ‘includes’, ‘including’, ‘having’ and their inflections and conjugates denote ‘including but not limited to’.

The term ‘may’ denotes an option which is either or not included and/or used and/or implemented, yet the option constitutes at least a part of the invention.

Unless the context indicates otherwise, referring to an object in the singular o form (e.g. “a thing” or “the thing”) does not preclude the plural form (e.g. “the things”).

The present invention has been described using descriptions of embodiments thereof that are provided by way of example and are not intended to limit the scope of the invention or to preclude other embodiments. The described embodiments comprise various features, not all of which are necessarily required in all embodiments of the invention. Some embodiments of the invention utilize only some of the features or possible combinations of the features. Alternatively and additionally, portions of the invention described or depicted as a single unit may reside in two or more separate entities that act in concert or otherwise to perform the described or depicted function. Alternatively and additionally, portions of the invention described or depicted as two or more separate physical entities may be integrated into a single entity to perform the described/depicted function. Variations related to one or more embodiments may be combined in all possible combinations with other embodiments.

In the specifications and claims, unless particularly specified otherwise, when operations or actions or steps are recited in some order, the order may be varied in any practical manner.

Terms in the claims that follow should be interpreted, without limiting, as characterized or described in the specification.

What we claim is:

1. A fastenable pelvic splint, comprising:
(a) a buckle comprising a contractible ratchet mechanism and a fastening handle; and
(b) a flexible band adapted to be connected to the buckle at two opposite ends of the buckle and to circumscribe the pelvis.

2. The splint according to claim 1, adapted to be fastened around the pelvic by contracting the distance between the two opposite ends of the buckle using the ratchet mechanism.

3. The splint according to claim 2, wherein the splint is adapted to be fastened by a motion of the handle and wherein the handle is operable using a single hand.

4. The splint according to claim 1, wherein the buckle further comprises a locking mechanism adapted to lock the splint in a fasten position.

5. The splint according to claim 1, wherein the buckle further comprises a release trigger adapted to loosen the splint.

6. The splint according to claim 1, wherein the band is adapted to be removably connected to the buckle.

7. The splint according to claim 1, wherein the band comprises linkage formations along at least a portion of the length of the band, wherein the linkage formations are adapted to connect the band to the buckle.

8. The splint according to claim 7, wherein the buckle comprises structures adapted to mate with the linkage formations of the band.

9. The splint according to claim 7, wherein the buckle comprises buttons and wherein the linkage formations of the band comprise slits, wherein the buttons are adapted to fit to the slits thereby connect the band to the buckle.

10. The splint according to claim 1, wherein the splint is substantially x-ray transparent.

11. The splint according to claim 1, wherein the splint is disposable.

12. A kit for a pelvic splint, comprising:
(a) at least one buckle comprising a contractible ratchet mechanism and a fastening handle; and
(b) at least one flexible band adapted to be connected to the buckle at two opposite ends of the buckle and to circumscribe the pelvis.

13. The kit according to claim 12, wherein the splint is adapted to be fastened around the pelvic by contracting the distance between the two opposite ends of the buckle using the ratchet mechanism.

14. The kit according to claim 12, wherein the splint is adapted to be fastened by a motion of the handle and wherein the handle is operable using a single hand.

15. The kit according to claim 12, wherein the buckle further comprises a locking mechanism adapted to lock the splint in a fasten position.

16. The kit according to claim 12, wherein the buckle further comprises a release trigger adapted to loosen the splint.

17. The kit according to claim 12, wherein the band is adapted to be removably connected to the buckle.

18. The kit according to claim 12, wherein the band comprises linkage formations along at least a portion of the length of the band, wherein the linkage formations are adapted to connect the band to the buckle.

19. The kit according to claim 18, wherein the buckle comprises structures adapted to mate with the linkage formations of the band.

20. The kit according to claim 18, wherein the buckle comprises buttons and wherein the linkage formations of the band comprise slits, wherein the buttons are adapted to fit to the slits thereby connect the band to the buckle.

21. The kit according to claim 12, wherein the splint is substantially x-ray transparent.

22. The kit according to claim 12, wherein the splint is disposable.
